(19) United States
(12) Patent Application Publication
(10) Pub. No.: US 2016/0000387 A1
(43) Pub. Date: Jan. 7, 2016

(54) BODY SUPPORT WITH ANATOMICAL APERTURE

(71) Applicant: Indiana University Research and Technology Corp., Indianapolis, IN (US)

(72) Inventors: Jeffrey Buchsbaum, Bloomington, IN (US); Victor Simoneaux, JR., Indianapolis, IN (US); Archana Gautaum, Indianapolis, IN (US); Amy Sandefur, Indianapolis, IN (US)

(21) Appl. No.: 14/789,009

(22) Filed: Jul. 1, 2015

Related U.S. Application Data
(60) Provisional application No. 62/020,132, filed on Jul. 2, 2014.

(51) Int. Cl. A61B 6/04; A61B 19/02; A61B 5/055; A61B 19/00

(52) U.S. Cl. CPC A61B 6/0435; A61B 19/40; A61B 19/0248; A61B 5/0555; A61B 2019/4036; A61B 2019/0255

(57) ABSTRACT

A body support comprising a support surface with at least one anatomical aperture configured to allow at least one appendage to protrude through the at least one anatomical aperture for a medical treatment is disclosed. Additionally, a method for positioning a user on the body support is also disclosed.

### CROSS REFERENCE TO RELATED APPLICATIONS

The present application claims the benefit of and priority to, under 35 U.S.C. §119(e), U.S. Provisional Application Ser. No. 62/020,132, filed Jul. 2, 2014, entitled “BODY SUPPORT WITH ANATOMICAL RECESS,” which is hereby incorporated herein by reference in its entirety for all that it teaches and for all purposes.

### FIELD

The present disclosure relates generally to a body support with an anatomical aperture. More particularly, the present disclosure relates to a body support with an anatomical aperture for treatment of cancer and a method of positioning a patient for such treatment.

### BACKGROUND

Early and effective detection and treatment of cancer are necessary to prevent serious health deterioration and death in cancer patients. According to the Centers for Disease Control and Prevention, cancer was the second leading cause of death in both women and men behind heart disease in 2010. In addition to certain types of skin cancer, breast cancer is the most common cancer in women, regardless of race or ethnicity. Prostate cancer is the most common form of cancer in men.

One effective means of cancer treatment is proton beam therapy, which can help reduce or eliminate tumor growth. The positioning of the patient's body when treating a patient with proton beam therapy is important because the proton beam should only be targeted at the anatomical region of the body with cancer. Precise targeting of the cancer cells with proton beam therapy helps to avoid any unintended consequences to surrounding or adjacent healthy tissues. For example, when treating breast cancer with proton beam therapy, the proton beam should treat the entire breast, but stop before interacting with the patient's lungs or ribs.

### SUMMARY

As discussed above, for some treatments, a patient's body positioning is important to reduce the propagation of the treatment to adjacent healthy cells. For example, when treating cancerous tumor cells in the breast with proton beam treatment, the entire breast should be treated, but the proton beam should not propagate to surrounding healthy cells in lung tissue or ribs, where damage could occur to the healthy cells. The embodiments herein provide a solution to this problem by disclosing a body support that comprises a support surface that includes at least one anatomical aperture. A user (e.g., a patient) can be positioned on the body support so that the unhealthy body part(s) can protrude into the at least one anatomical aperture, thereby creating separation of the unhealthy tissue from nearby healthy tissue. Possible medical treatments that can be used on the unhealthy tissue can include, but are not limited to, proton beam therapy, treatment planning using a CT (computerized tomography) or treatment planning using a MR (Magnetic Resonance) scanner.

As an example, if a patient with breast cancer were positioned face down in a prone position on the body support provided herein with an anatomical aperture surrounding the breasts, gravity would cause the breasts to hang downwardly. While the breasts (or, in other instances, other bodily appendages such as limbs or genitals) are hanging downwardly below the support through the anatomical aperture, there is separation created between the breasts and chest. The proton beam therapy (or other cancer treatment) can then target the cancerous breast without propagating as easily to healthy cells in the lungs or ribs.

Exemplary body supports and exemplary methods for positioning a user on the body support include the following.

In Example 1, a body support comprises: a support surface with at least one anatomical aperture configured to allow at least one appendage to protrude through the at least one anatomical aperture for a medical treatment.

In Example 2, the body support according to Example 1 is provided, wherein an anatomical aperture of the at least one anatomical aperture comprises three sides.

In Example 3, the body support according to either Example 1 or 2 is provided, wherein an anatomical aperture of the at least one anatomical aperture is positioned proximate a first end of the body support to allow for a user's chest to protrude through the anatomical aperture when the user lies on the support surface in a prone position with the user's head positioned proximate the first end.

In Example 4, the body support according to any of Examples 1-3 is provided, further comprising one or more handles on a first side and a second side of the body support, wherein the second side is positioned opposite to and substantially parallel with the first side.

In Example 5, the body support according to any of Examples 1-4 is provided, further comprising at least one opening, wherein the at least one opening is configured to receive at least one support leg to support the support surface.

In Example 6, the body support according to any of Examples 1-5 is provided, wherein the support surface further comprises ergonomic contours to guide proper positioning of a user's body in a prone position.

In Example 7, the body support according to any of Examples 1-6 is provided, wherein the medical treatment is selected from the group consisting of: proton beam therapy for a cancerous appendage, treatment planning using a CT (computerized tomography) scanner, and treatment planning using a MR (Magnetic Resonance) scanner.

In Example 8, a method for positioning a user on a body support comprising: positioning the user in a prone position on a support surface of a body support so that an appendage of the user protrudes through an anatomical aperture of the body support; and performing a medical treatment that targets the appendage of the user.

In Example 9, the method according to Example 8 is provided, wherein the anatomical aperture comprises three sides.

In Example 10, the method according to either Examples 8 or 9 is provided, wherein the anatomical aperture is positioned proximate a first end of the body support to allow for a user's chest to protrude through the anatomical aperture when the user lies on the support surface in a prone position with the user's head positioned proximate the first end.

In Example 11, the method according to any of Examples 8-10 is provided, wherein performing a medical treatment includes performing a treatment selected from the group consisting of: proton beam therapy for a cancerous appendage, treatment planning using a CT (computerized tomography) scanner, and treatment planning using a MR (Magnetic Resonance) scanner.

In Example 12, the method according to any of Examples 8-11 is provided, wherein the body support comprises one or more handles on a first side and a second side of the body support, wherein the second side is positioned opposite to and substantially parallel with the first side.

In Example 13, the method according to any of Examples 8-12 is provided, wherein the support surface comprises ergonomic contours to guide proper positioning of the user's body in a prone position.

In Example 14, a body support with an anatomical aperture is provided, the body support comprising: a support surface with a first end and a second end, wherein said first and second ends are of substantially equal length and substantially parallel; a first side, wherein said first side is disposed between the first end and the second end and wherein said first side is substantially perpendicular to the first and second ends; a second side, wherein said second side is disposed opposite the first side and between the second end and a third side, wherein said third side is disposed substantially perpendicular to the second side; a fourth side, wherein said fourth side is substantially parallel to the first side and is disposed perpendicularly between the third side and a fifth side, wherein the fifth side is substantially parallel to the third side; and a sixth side, wherein said sixth side is disposed substantially perpendicularly between the first end and the fifth side and is substantially parallel to the first side, where the third, fourth, and fifth sides form an anatomical aperture, and wherein the anatomical aperture is configured to allow an appendage to protrude into the anatomical aperture.

In Example 15, the body support according to Example 14 is provided, wherein the appendage protrudes into and through the anatomical aperture.

In Example 16, the body support according to either Example 14 or 15 is provided, wherein the length of the sixth side is less than the length of the second side, thereby forming an anatomical aperture positioned proximate a first end of the body support to allow for a user's chest to hang downwardly through the anatomical aperture when the user lies on the support surface in a prone position with the user's head positioned proximate the first end.

In Example 17, the body support according to any of Examples 14-16 is provided, wherein the body support is configured for conducting a medical treatment with a user when the user is in a prone position, wherein the medical treatment is selected from the group consisting of: proton beam therapy for a cancerous appendage, treatment planning using a CT (computerized tomography) scanner, and treatment planning using a MR (Magnetic Resonance) scanner.

In Example 18, the body support according to any of Examples 14-17 is provided, further comprising one or more handles on the first side and the second side of the body support.

In Example 19, the body support according to any of Examples 14-18 is provided, further comprising at least one opening, wherein the at least one opening is configured to receive at least one support leg to support the support surface.

In Example 20, the body support according to any of Examples 14-19 is provided, wherein the support surface has ergonomic contours to guide proper positioning of the user's body in a prone position.

As used herein, the terms “couple” and “coupling” or “connect” and “connected” as used herein may refer to any permanent or removable connection for medical parts known in the art including, but not limited to, connections with hinges, actuators, hydraulics, bolts, screws, threads, magnets, electro-magnets, adhesives, friction grips, welds, snaps, clips, etc.

### BRIEF DESCRIPTION OF THE DRAWINGS

The features of this disclosure, and the manner of attaining them, will become more apparent, and the disclosure itself will be better understood by reference to the following description of embodiments of the disclosure taken in conjunction with the accompanying drawings.

FIG. 1 is a top plan view of an exemplary body support with at least one anatomical aperture.

FIG. 2 is a side plan view of the exemplary body support of FIG. 1.

FIGS. 3A-3B are front plan views of the exemplary body support of FIG. 1.

FIG. 4 is a perspective view of the exemplary body support of FIG. 1 in use with a user.

FIG. 5 is a side plan view of the exemplary body support of FIG. 1 with an optional co-planar insert.

FIG. 6 is a flow diagram of an exemplary method for positioning a user on a body support.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplifications set out herein illustrate an exemplary embodiment of the disclosure, in one form, and such exemplifications are not to be construed as limiting the scope of the disclosure in any manner.

### DETAILED DESCRIPTION

The embodiments disclosed herein are not intended to be exhaustive or limit the disclosure to the precise form disclosed in the following detailed description. Rather, the embodiments are chosen and described so that others skilled in the art may utilize their teachings.

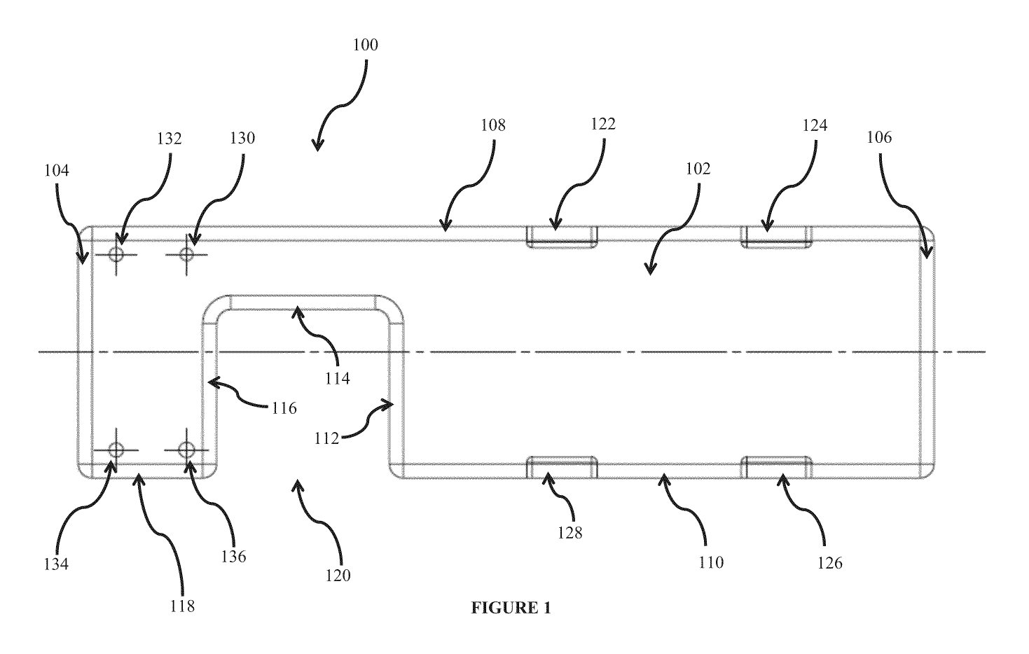

FIG. 1 is a top plan view of an exemplary body support 100 with a support surface 102 that includes at least one anatomical aperture 120. In one embodiment, body support 100 is easily-movable and light-weight such that it could be carried and moved by a person to a needed location, yet is strong enough to support a person lying on body support 100 in a prone position. In other embodiments, body support 100 is stationary and supported by supports that are stationary relative to a floor or other surface. Still in other embodiments, body support 100 may extend from and retract into a wall, closet, container, or other enclosing space by springs, hinges, gears, hydraulics, or any other means known in the art such that when body support 100 is not in use, it is not visible. Body support 100 can be made of metal, plastic, foam, composite material or any other combination of materials. In some embodiments, body support 100 can include material configured to block certain types of medical treatment. This may help in isolating the healthy tissue from the unhealthy tissue that is receiving the medical treatment.

Body support 100 includes a support surface 102, which in the embodiment shown is substantially flat or planar. In other embodiments, support surface 102 can be bent or curved upwardly and/or downwardly with one or more bends or contours. In some embodiments, the contours or bends allow for certain ergonomic placement of the human body when in a prone position on support surface 102. For example, channels, grooves or contours could ergonomically support the arms, legs, or other body parts when a person is in a prone position on support surface 102. Support surface 102 can be made of any material sufficient to support a human body including, but not limited to, metal, plastic, foam, composite material, or any combination of materials.

Support surface 102 optionally is equipped with pads, pillows, or other comfort devices for a human body when in a prone position. Additionally, as shown support surface 102 is a continuous surface; however, in other embodiments support surface 102 may include more than one surface removably or permanently coupled by hinges, snaps, hydraulics, actuators, or any other sufficient connecting means to create a stable surface to support a human body in the prone position. Support surface 102 may be solid or may be substantially hollow such that cords, wires, tubes, or other elements required in medical treatments may pass through support surface 102.

Body support 100 further comprises a first end 104 and a second end 106. In the embodiment shown, first end 104 and second end 106 are of substantially equal length and substantially parallel; however, in other embodiments first end 104 and second end 106 may be of differing lengths and/or not substantially parallel.

First end 104 and second end 106 may be permanently or removably coupled to body support 100, and optionally have grips, grooves, handles, or holes for lifting or moving body support 100 to a desired position. Furthermore, first end 104 and second end 106 may be curved or square around the edges. In other embodiments, first end 104 and second end 106 have rests, grooves, contours, pillows, or other means to increase the comfort of a user lying in a prone position on body support 100. For example, if a user were to lie in a prone position on body support 100 with head facing toward first end 104 and feet toward second end 106, a pillow or other comfort device might be placed at or near first end 104 and a similar pillow or other comfort device might be placed at or near second end 106. First end 104 and second end 106 might be solid or might be substantially hollow to allow for the passage of cords, wires, tubes or other elements required in medical treatments.

Support further comprises a first side 108. In some embodiments, first side 108 is disposed in a substantially straight fashion between first end 104 and second end 106, as shown. In these embodiments, first side 108 is substantially perpendicular to the first and second ends. In some other embodiments, first side 108 might be contoured or grooved in any fashion between first end 104 and second end 106 to ergonomically support a user lying face-down on body support 100 and to increase the user's comfort. Further, such optional grooves or contours in first side 108 might be used to increase the ease of moving and positioning body support 100 and to increase the ease of storing body support 100. First side 108 might be solid or might be substantially hollow to allow for the passage of cords, wires, tubes or other elements required in medical treatments.

In some embodiments, first side 108 includes handles 122, 124, which help a user move and position body support 100. Handles 122,124 might be grooves disposed on or within first side 108, or handles 122,124 might be removable or permanent straps attached to first side 108. In other embodiments, handles 122, 124 might include a combination of straps and grooves. More or fewer handles disposed in alternative locations on first side 108 are also envisioned.

In some alternative embodiments, first side 108 is coupled to a wall or stationary support by hinges, actuators, springs, or other means that would allow for easy storage of body support 100 when not in use. For example, when body support 100 is in use it might be parallel to and above a floor or other flat surface, supported by means sufficient to hold a human body lying in the prone position. When not in use, body support 100 might be parallel and in contact with a wall or closet. One can envision a doctor or other user manually, or remotely by wired or wireless means, unfolding body support 100 from a wall or closet for a user to lie down, and retracting body support 100 back into a wall or closet when not in use. Such coupling means for storage could also be applied to either first end 104 or second end 106, and also could be applied to any of the sides discussed further below.

Body support 100 further comprises a second side 110, which is disposed opposite first side 108 and between second end 106 and a third side 112, as shown. In some embodiments, second side 110 is substantially straight and perpendicular to second end 106 and third side 112. In some other embodiments, second side 110 might be contoured or grooved in any fashion between second end 106 and third side 112 to ergonomically support a user lying face-down on body support 100 and to increase the user's comfort. Further, such optional grooves or contours in second side 110 might be used to increase the ease of moving and positioning body support 100 and to increase the ease of storing body support 100. Second side 110 might be solid or might be substantially hollow to allow for the passage of cords, wires, tubes or other elements required in medical treatments.

In some embodiments, second side 110 includes handles 126, 128, which help a user move and position body support 100. Handles 126,128 might be grooves disposed on or within second side 110, or handles 126,128 might be removable or permanent straps attached to second side 110. In other embodiments, handles 126, 128 might include a combination of straps and grooves. More or fewer handles are also envisioned in alternative locations on second side 110.

As stated above, body support 100 includes at least one anatomical aperture 120. The anatomical aperture 120 is configured to allow at least one appendage to protrude into the anatomical aperture 120 for a medical treatment. In some embodiments, the appendage protrudes into and through the anatomical aperture 120. Exemplary medical treatments that can be performed on the appendage protruding into the anatomical aperture 120 can include, but are not limited to, proton beam therapy for a cancerous appendage, treatment planning using a CT (computerized tomography) scanner, and treatment planning using a MR (Magnetic Resonance) scanner.

In the embodiment shown, there is one anatomical aperture; however, in other embodiments there could be more anatomical apertures. For example, if a male were to lie on body support 100 in a prone position with his head positioned toward first end 104 and his feet toward second end 106, there may be anatomical aperture 120 to accommodate his chest and a second anatomical aperture (not shown) positioned between aperture 120 and second end 106 to accommodate his midsection. In such a way, anatomical aperture 120, and any additional anatomical apertures, allow for appendages of the human body to hang downwardly by gravity below a plane formed by support surface 102 (discussed further below). Thus, cancerous appendages can hang downwardly in an open space, in some embodiments a treatment space, between body support 100 and a floor or lower surface.

In some embodiments, the anatomical aperture 120 is positioned proximate the first end 104 of the body support 100. This configuration can allow a user's chest to protrude through the anatomical aperture 120 when the user lies on the support surface 102 in a prone position with the user's head positioned proximate the first end 102. The user's chest can then be targeted using a medical treatment, such as proton beam therapy.

In some embodiments, the anatomical aperture 120 is formed in body support 100 by three sides, third side 112, a fourth side 114 and a fifth side 116, as shown in FIG. 1.

In some embodiments, third side 112 is positioned substantially parallel to first end 104 and second end 106, and substantially perpendicular to first side 108, second side 110, and fourth side 114. Third side 112 need not be substantially straight as shown and, in other embodiments, can be grooved, padded, contoured, or otherwise ergonomically favorably shaped for a user's torso when a user lies face-down on support surface 102 in a prone position. Third side 112 optionally has handles or straps, similar to those discussed previously, for easy positioning and movement of body support 100. Third side 112 might be solid or might be substantially hollow to allow for the passage of cords, wires, tubes or other elements required in medical treatments.

In some embodiments, fourth side 114 is positioned inwardly towards first side 108, and away from second side 110. In some embodiments, fourth side 114 is substantially parallel to first side 108 and second side 110 and is disposed perpendicularly relative to first end 104, second end 106, and third side 112. Fourth side 114 need not be substantially straight as shown and, in other embodiments, can be grooved, padded, contoured, or otherwise ergonomically favorably shaped for a user's torso when a user lies face-down on support surface 102 in a prone position. Fourth side 114 optionally has handles or straps, similar to those discussed previously, for easy positioning and movement of body support 100. Fourth side 114 might be solid or might be substantially hollow to allow for the passage of cords, wires, tubes or other elements required in medical treatments.

In some embodiments, fifth side 116 is positioned substantially parallel relative to first end 104, second end 106, and third side 112. And, in some embodiments, fifth side 116 is positioned substantially perpendicular relative to first side 108 and second side 110. Fifth side 116 need not be substantially straight as shown and, in other embodiments, can be grooved, padded, contoured, or otherwise ergonomically favorably shaped for a user's torso when a user lies face-down on support surface 102 in a prone position. Fifth side 116 optionally has handles or straps, similar to those discussed previously, for easy positioning and movement of body support 100. Fifth side 116 might be solid or might be substantially hollow to allow for the passage of cords, wires, tubes or other elements required in medical treatments.

In some embodiments, body support 100 includes a sixth side 118, which is disposed substantially perpendicularly between first end 104 and fifth side 118, and is substantially parallel to first side 108, second side 110, and fourth side 114. Sixth side 118 need not be substantially straight as shown and, in other embodiments, can be grooved, padded, contoured, or otherwise ergonomically favorably shaped for a user's torso when a user lies face-down on support surface 102 in a prone position. Sixth side 118 optionally has handles or straps, similar to those discussed previously, for easy positioning and movement of body support 100. Sixth side 118 might be solid or might be substantially hollow to allow for the passage of cords, wires, tubes or other elements required in medical treatments.

While straight sides 112, 114, 116 are shown in FIG. 1, sides 112, 114, 116 do not have to be substantially straight, as described above. Moreover, anatomical aperture 120 may include more or fewer sides. For example, in some embodiments, anatomical aperture 120, or an alternatively disposed anatomical aperture, may include an appropriate number of sides to be substantially circular in shape, substantially oval-shaped, substantially c-shaped, substantially v-shaped, or any other shape providing a sufficient opening for an appendage to hang downwardly by gravity when a user lies in a prone position on support surface 102.

In some embodiments, support surface 102 includes openings 130, 132, 134, 136. In the embodiment shown, these openings pass entirely through support surface 102 and are positioned between first end 104, first side 108, fifth side 116, and sixth side 118. Such openings may be configured to receive support legs (not shown) that would provide support for support surface 102 in an upward direction relative to the floor. In other embodiments, there may be more or fewer openings than what is shown in FIG. 1, and they may be positioned anywhere convenient on support surface 102 to provide support to support surface 102 from the floor or other surface. Such openings 130, 132, 134, 136 need not pass entirely through support surface 102 and may only be visible from either a top side or bottom side of support surface 102. Openings 130, 132, 134, 136 may also provide one or more passages for cords, wires, tubes or other elements required in medical treatments to pass through support surface 102.

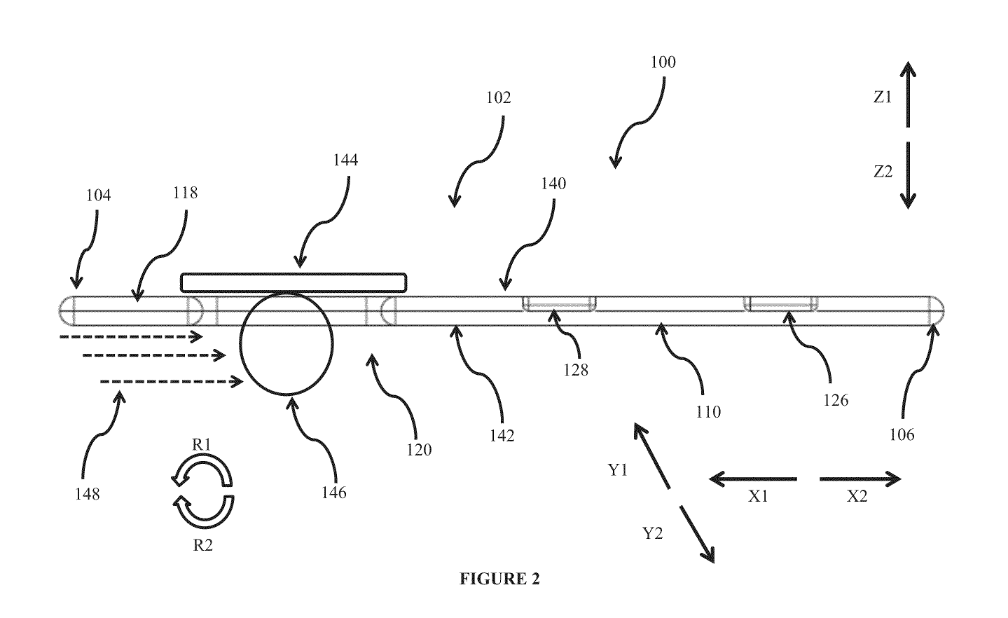

Referring to FIG. 2, a side plan view of exemplary body support 100 of FIG. 1 is shown. Support surface 102 has an upper surface 140 and a lower surface 142. The volume between upper surface 140 and lower surface 142 can be either substantially solid or substantially hollow to allow for passage of certain cords, wires, tubes, or other elements required in medical treatments. In the embodiment shown, support surface 102 is substantially flat and is a single unit; however, in other embodiments support surface 102 could include separate but coupled elements which bend or contour upwardly or downwardly.

The thickness of support surface 102 between upper surface 140 and lower surface 142 can vary but, in exemplary embodiments, should be of a thickness such that when a user lies in the prone position on upper surface 140, the user's appendages which require medical treatment hang below a plane formed by lower surface 142.

In some embodiments, body support 100 is movable in the X1, X2, Y1, Y2, Z1, Z2, R1, and R2 directions. For example, the height of body support 100 in the Z direction might be adjusted in the Z2 direction closer to a floor or surface when a user goes from a standing position adjacent to body support 100 to a prone position on upper surface 140 of support surface 102. Then, once the user is comfortably positioned on body support 100, it could be raised in the Z1 direction, or rotated in the R2 direction such that a doctor or other medical care provider would have access to the user's chest (or other appendage at another anatomical aperture). In one instance, if a user were positioned in a prone position on upper surface 140 and secured (possibly by straps or body-fitting ergonomic grooves) then body support 100 could be rotated in the R2 direction so that a doctor or care provider positioned near first end 104 could view the user's chest positioned in anatomical aperture 120.

For movement of body support 100, any combination of motors, actuators, hydraulics, pumps, or gears is envisioned. Body support 100 optionally has a user interface with controls for movement and a memory. Additionally, the movement of body support 100 in the aforementioned directions may be controlled wirelessly.

Block 144 represents a human body lying in the prone position on upper surface 140 of support surface 102. Cancerous appendage 146 is shown hanging downwardly by gravity through anatomical aperture 120 below lower surface 142. Appendage 146 can represent any appendage that is in need of treatment for cancer by means of proton beam therapy, or a similarly targeted beam therapy, and is capable of hanging downwardly by gravity through anatomical aperture 120. Appendage 146 might represent a breast with breast cancer or in other embodiments might represent male genitals with a type of genital cancer. Dotted arrows 148 represent proton beam therapy or a related targeted cancer therapy treating cancerous appendage 146. Proton beams 148 only interact with cancerous appendage 146 and do not interact with human body 144 which includes ribs, lungs, etc.

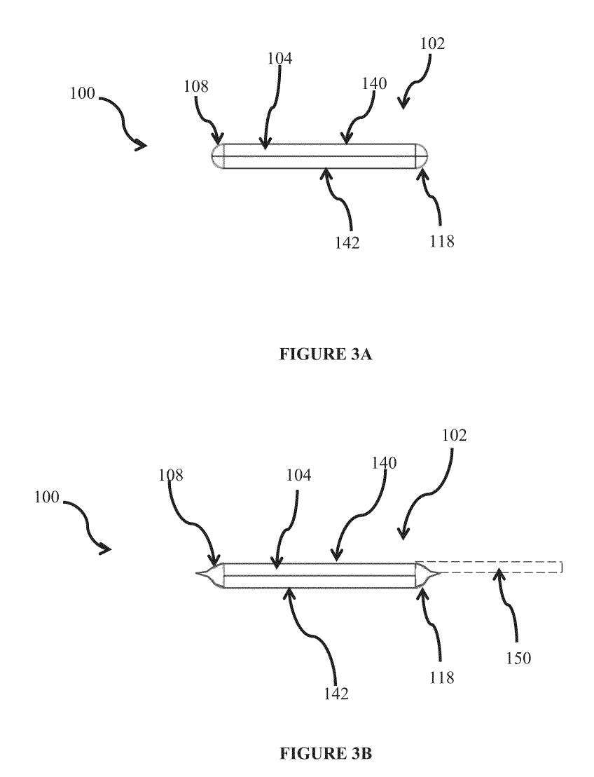

Referring now to FIG. 3A, a front plan view of exemplary body support 100 of FIG. 1 is shown. First end 104, and sides 108, 118 may be substantially hollow and optionally have outlets for allowing cords or tubes to pass through support surface 102. As shown, upper surface 140 and lower surface 142 are substantially flat or planar. In other embodiments, these surfaces may be contoured, grooved, bent, or positioned in a non-planar fashion.

In this embodiment, first side 108 and sixth side 118 are outwardly curved. In other embodiments, first side 108 and sixth side 118 may have square sides (not shown). In even other embodiments, first side 108 and sixth side 118 may have pointed sides, as shown in FIG. 3B. Pointed sides 108, 118 can be advantageous when there is secondary surface (e.g., secondary surface 150 in FIG. 3B) positioned next to the support surface 102. If sides 108, 118 are pointed, the secondary surface can be positioned co-planar with the support surface 102. In some embodiments, only one of the sides 108, 118 is pointed.

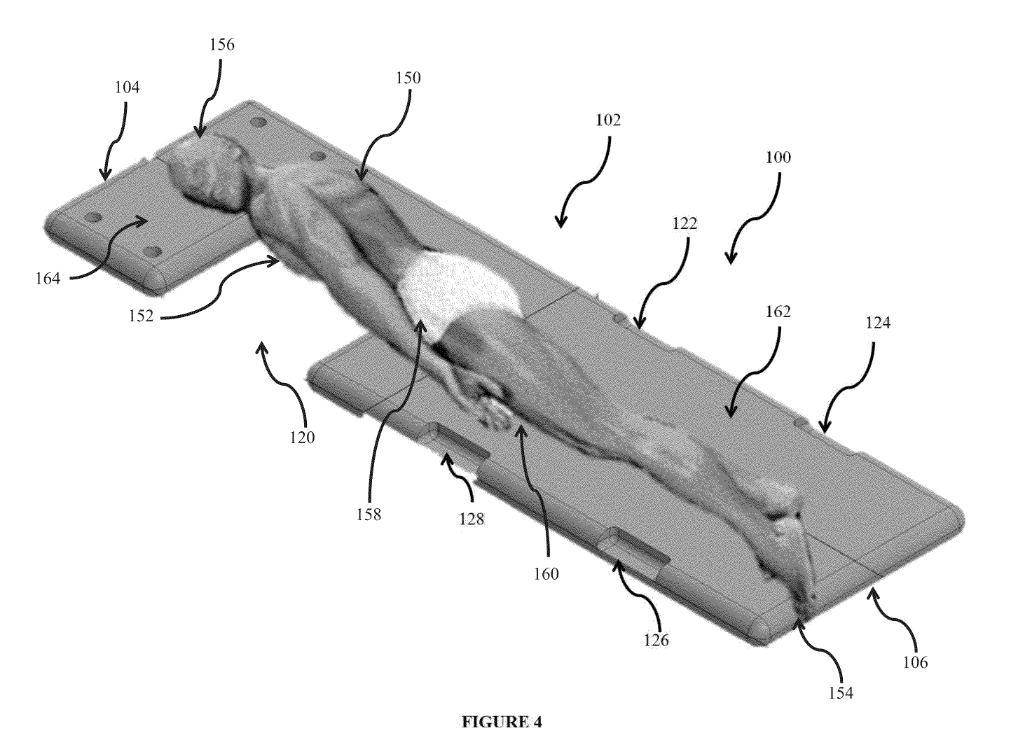

Referring now to FIG. 4, a perspective view of exemplary body support 100 of FIG. 1 is shown. A user 150 lies face downwardly in a prone position, while positioning chest 152 within anatomical aperture 120 to be accessible to a doctor and certain medical treatment below lower surface 142 of support surface 102. User 150 has feet 154 positioned near second end 106 and head 156 positioned near first end 104. Lower body 160 of user 150 is supported by lower body support portion 162 of support surface 102. User 150 has head 156 supported by upper body support portion 164 of support surface 102. In alternative embodiments, user 150 might have midsection 158 positioned above an additional anatomical aperture (not pictured) such that additional appendages could hang below lower surface 142 for medical treatment.

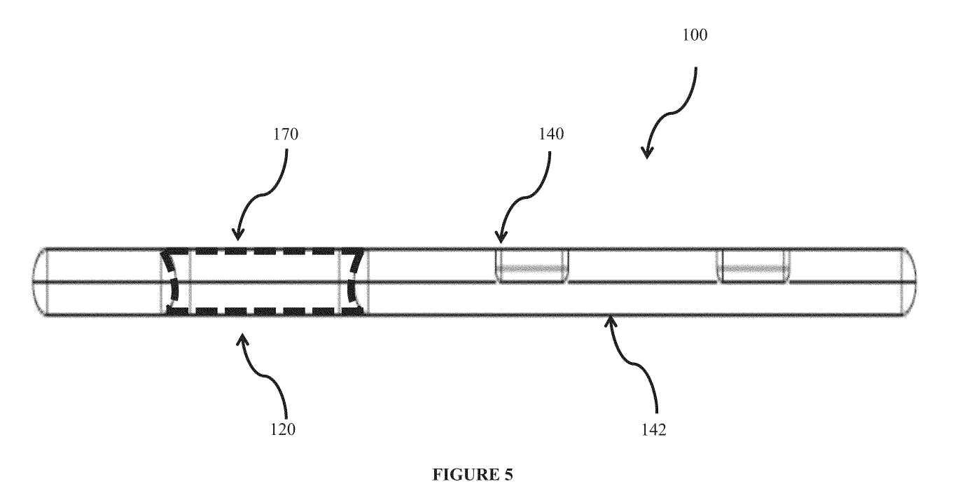

Referring now to FIG. 5, a side plan view of the exemplary body support 100 of FIG. 1 with an optional co-planar insert 170 is shown. Co-planar insert 170 may be made of the same or similar material as body support 100, or co-planar insert 170 may be made of different material, such as a softer, more supportive, or grooved material to fit under a user's chest. Co-planar insert 170 would allow for a user to lie down on body support 100 fully, as shown in FIG. 4, before having the user's chest cavity extend downwardly into anatomical aperture 120, when co-planar insert 170 is removed. Co-planar insert 170 may fill all or substantially all of anatomical aperture 120 or co-planar insert 170 may only fill a portion of anatomical aperture 120 or have grooves or apertures itself.

In the embodiment shown in FIG. 5, co-planar insert 170 is co-planar with support surface 102 at upper surface 140 and lower surface 142. However, in other embodiments, insert 170 may be co-planar with only one surface. Co-planar insert 170 may be removed manually by hand, or according to a computer program.

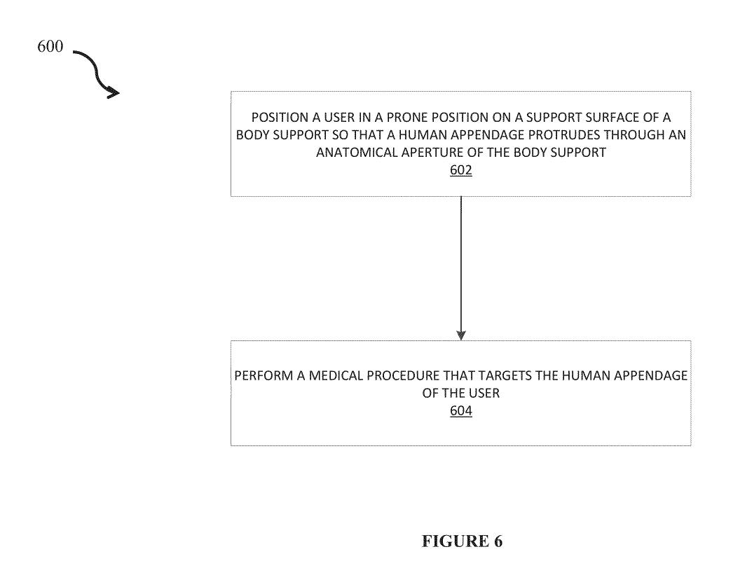

FIG. 6 is a flow diagram of an exemplary method 600 for positioning a user on a body support. Method 600 comprises positioning a user in a prone position on a support surface of a body support so that an appendage of the user protrudes through an anatomical aperture of the body support (block 602). As a result, it may be possible to target the user's chest (or other appendage) with a medical treatment, but not affect healthy tissue in the lungs, healthy cells in the ribs or other proximate tissue.

The body support, the support surface and the anatomical aperture can have some or all of the same characteristics of the body support 100, the support surface 102 and the anatomical aperture 120 described above in FIGS. 1-5, respectively. For example, in some embodiments, the support surface comprises ergonomic contours to guide proper positioning of the user's body in a prone position. In some embodiments, the body support can include a first end, a second end, a first side, a second side and a sixth side, wherein the second side and the sixth side are positioned opposite to and substantially parallel with the first side. Additionally, the body support can include one or more handles on the first side and the second side.

Moreover, in some embodiments, the anatomical aperture of the body support can comprise three sides (referred to as a third side, a fourth side and a fifth side, as referred to in FIGS. 1-5 above) and be located proximate the first end of the body support to allow for a user's chest to protrude through the anatomical aperture when the user lies on the support surface in a prone position with the user's head positioned proximate the first end. The third side, the fourth side and the fifth side can include one or more handles, as well.

Method 600 further comprises performing a medical treatment that targets the appendage of the user (block 604). The medical treatment can include, but is not limited to, the following: proton beam therapy for a cancerous appendage, treatment planning using a CT (computerized tomography) scanner, and treatment planning using a MR (Magnetic Resonance) scanner.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A body support comprising:
a support surface with at least one anatomical aperture configured to allow at least one appendage to protrude through the at least one anatomical aperture for a medical treatment.

2. The body support according to claim 1, wherein an anatomical aperture of the at least one anatomical aperture comprises three sides.

3. The body support according to claim 1, wherein an anatomical aperture of the at least one anatomical aperture is positioned proximate a first end of the body support to allow for a user's chest to protrude through the anatomical aperture when the user lies on the support surface in a prone position with the user's head positioned proximate the first end.

4. The body support according to claim 1, further comprising one or more handles on a first side and a second side of the body support, wherein the second side is positioned opposite to and substantially parallel with the first side.

5. The body support according to claim 1, further comprising at least one opening, wherein the at least one opening is configured to receive at least one support leg to support the support surface.

6. The body support according to claim 1, wherein the support surface further comprises ergonomic contours to guide proper positioning of a user's body in a prone position.

7. The body support according to claim 1, wherein the medical treatment is selected from the group consisting of: proton beam therapy for a cancerous appendage, treatment planning using a CT (computerized tomography) scanner, and treatment planning using a MR (Magnetic Resonance) scanner.

8. A method for positioning a user on a body support comprising:
positioning a user in a prone position on a support surface of a body support so that an appendage of the user protrudes through an anatomical aperture of the body support; and
performing a medical treatment that targets the appendage of the user.

9. The method according to claim 8, wherein the anatomical aperture comprises three sides.

10. The method according to claim 8, wherein the anatomical aperture is positioned proximate a first end of the body support to allow for a user's chest to protrude through the anatomical aperture when the user lies on the support surface in a prone position with the user's head positioned proximate the first end.

11. The method according to claim 8, wherein performing a medical treatment includes performing a treatment selected from the group consisting of: proton beam therapy for a cancerous appendage, treatment planning using a CT (computerized tomography) scanner, and treatment planning using a MR (Magnetic Resonance) scanner.

12. The method according to claim 8, wherein the body support comprises one or more handles on a first side and a second side of the body support, wherein the second side is positioned opposite to and substantially parallel with the first side.

13. The method according to claim 8, wherein the support surface comprises ergonomic contours to guide proper positioning of the user's body in a prone position.

14. A body support with an anatomical aperture, the body support comprising:
a support surface with
a first end and a second end, wherein said first and second ends are of substantially equal length and substantially parallel;
a first side, wherein said first side is disposed between the first end and the second end and wherein said first side is substantially perpendicular to the first and second ends;
a second side, wherein said second side is disposed opposite the first side and between the second end and a third side, wherein said third side is disposed substantially perpendicular to the second side;
a fourth side, wherein said fourth side is substantially parallel to the first side and is disposed perpendicularly between the third side and a fifth side, wherein the fifth side is substantially parallel to the third side; and
a sixth side, wherein said sixth side is disposed substantially perpendicularly between the first end and the fifth side and is substantially parallel to the first side, where the third, fourth, and fifth sides form an anatomical aperture, and wherein the anatomical aperture is configured to allow an appendage to protrude into the anatomical aperture.

15. The body support of claim 14, wherein the appendage protrudes into and through the anatomical aperture.

16. The body support according to claim 14, wherein a length of the sixth side is less than a length of the second side, thereby forming an anatomical aperture positioned proximate a first end of the body support to allow for a user's chest to hang downwardly through the anatomical aperture when the user lies on the support surface in a prone position with the user's head positioned proximate the first end.

17. The body support according to claim 14, wherein the body support is configured for conducting a medical treatment with a user when the user is in a prone position, wherein the medical treatment is selected from the group consisting of: proton beam therapy for a cancerous appendage, treatment planning using a CT (computerized tomography) scanner, and treatment planning using a MR (Magnetic Resonance) scanner.

18. The body support according to claim 14, further comprising one or more handles on the first side and the second side of the body support.

19. The body support according to claim 14, further comprising at least one opening, wherein the at least one opening is configured to receive at least one support leg to support the support surface.

20. The body support according to claim 14, wherein the support surface has ergonomic contours to guide proper positioning of the user's body in a prone position.
